(19) United States
(12) Patent Application Publication
(10) Pub. No.: US 2006/0003969 A1
(43) Pub. Date: Jan. 5, 2006

(54) Compositions and methods for treating pathological infections

(76) Inventor: Madhusudan P. Manandhar, Monsey, NY (US)

Correspondence Address: Barry H. Jacobsen;Legal Division, Warmer-Lambert Company, LLC, 201 Tabor Road, Morris Plains, NJ, 07950, US

(21) Appl. No.: 11/172,285

(22) Filed: Jun. 30, 2005

Related U.S. Application Data
(60) Provisional application No. 60/589,215, filed on Jul. 2, 2004.
(60) Provisional application No. 60/590,813, filed on Jul. 23, 2004.

(51) Int. Cl. A61K 31/66; A61K 31/12

(52) U.S. Cl. 514/125; 514/675

(57) ABSTRACT

The present invention relates to compositions and methods for treating pathological infections. An embodiment of the present invention is directed to a topical composition useful for the treatment of yeast, fungal or bacterial infections of a patient in need of such treatment which includes a pharmaceutically effective amount of an anti-yeast, anti-fungal or antibacterial pharmaceutically active agent; and a pharmaceutically acceptable carrier.

This application claims priority to U.S. provisional patent application No. 60/589,215, filed Jul. 2, 2005 and U.S. provisional patent application No. 60/590,813, filed Jul. 23, 2005.

### FIELD OF THE INVENTION

The present invention relates to treatments of pathological infections. More particularly, the present invention relates to treatment of nail, skin and mucous membrane for fungal, yeast and bacterial infections.

### DESCRIPTION OF RELATED ART

Yeast, bacteria and fungi can cause infections in the skin, hair and nails and can feed on keratinized nail tissue. Such infections can cause parts of skin and nails to thicken, discolor, disfigure and split. In some instances, these infections can cause pressure, irritation and pain on the skin and in the nail area. Infections such as onychomycosis are believed to be from fungi or dermatophytes, such as Trichophyton rubrum and Trichophyton mentagrophytes. Onychomycosis may also be caused by yeast, such as candida albicans or candida parapsilosis. Paronychia infections exhibit similar symptoms to onychomycosis and may be caused by bacteria such as staphylococcus, streptococcus, and pseudomonas.

Yeast, fungal and bacterial infections of the skin and nails are particularly common in patients with diabetes because blood circulation is poor in the extremities, which compromises one's ability to fight infections. Diabetic patients have to be particularly careful of such infections and may not be able to take traditional therapies due to their diabetic condition.

Bacterial and fungal infections are difficult to treat. Traditional topical therapies often do not penetrate the nail plate and do not eradicate the infection in and under the nail bed. Oral medication therapies are associated with potentially harmful side effects such as elevated liver enzymes, gastrointestinal disorders and skin rashes and may require expensive medical intervention and laboratory tests. Other treatment options include surgical removal of the nail or drilling of holes in the nail to allow penetration of topical treatments, however, both are extremely painful.

The rate of recurrence of fungal and bacterial infections of the skin, nail and mucous membranes remains high with traditional therapies. Accordingly, there remains a need for effective treatment of yeast, bacterial and fungal infections of the nail, skin and mucous membranes.

### SUMMARY

In the present invention, carbohydrates have been found to be effective in treating pathological infections such as fungal, yeast and bacterial infections. Examples of useful carbohydrates include but are not limited to sugars, such as aldehydes and ketones and complex carbohydrates. Useful sugars include but are not limited to aldehydes and ketones and combinations thereof. Useful ketones, include hydroxyketones such as dihydroxyacetone, isomers, enantiomers and derivatives thereof. Useful aldehydes include glyceraldehydes, isomers, enantiomers and derivatives thereof.

An embodiment of the present invention provides a topical anti-yeast, anti-fungal or antibacterial composition which includes a pharmaceutically effective amount of a pharmaceutically active agent selected from the group consisting of ketones, aldehydes and enantiomers, derivatives and combinations thereof and a pharmaceutically acceptable carrier. Another embodiment of the present invention is directed to a topical composition useful for the treatment of yeast, fungal or bacterial infections of a patient in need of such treatment which includes a pharmaceutically effective amount of an anti-yeast, anti-fungal or antibacterial pharmaceutically active agent including ketones, aldehydes and/or enantiomers, derivatives and combinations thereof; and a pharmaceutically acceptable carrier. A patient includes animals, mammals and humans.

Another embodiment provides for a composition including at least one first pharmaceutically effective amount of a first pharmaceutically active agent, including, dihydroxyacetone, dihydroxyacetone phosphate, glyceraldehyde, 3-phosphoglyceraldehyde and enantiomers, derivatives and combinations thereof and at least one second pharmaceutically active agent in a pharmaceutically effective amount including terbinafine, fluconazole, micanozole, doconosol and a derivative, enantiomer, pharmaceutically acceptable salt and combination thereof; and a pharmaceutically acceptable carrier.

A further aspect of the present invention provides a method for the treatment of bacterial, yeast and fungal infections including administering a pharmaceutically effective amount of a pharmaceutically active agent selected from the group consisting of a ketones, aldehydes and derivatives, enantiomers and combinations thereof to a patient in need of such treatment. The pharmaceutically active agent may be administered topically to infected skin, nail or mucous membranes. Yet a further embodiment of the present invention provides a method for the treatment of bacterial, yeast and fungal infections including administering a pharmaceutically effective amount of a ketone or aldehyde to a patient in need of such treatment. Still a further embodiment provides for a method for the treatment of pain associated with fungal, yeast and bacterial infections including administering a pharmaceutically effective amount of a ketone or aldehyde to a patient in need of such treatment. Useful ketones and aldehydes include dihydroxyacetone, glyceraldehydes, isomers, enantiomers and combinations thereof. In another embodiment, a method provides for concurrently administering, either topically or orally, at least one additional or ‘second’ pharmaceutically active agent including but not limited to terbinafine, miconazole, fluconazole, doconosol and a derivative, enantiomers, pharmaceutically acceptable salts and combinations thereof.

Another embodiment of the present invention provides a topical anti-fungal, anti-yeast and/or antibacterial composition which includes a pharmaceutically effective amount of a carbohydrate, including a sugar, and a pharmaceutically or cosmeceutically acceptable carrier. Another embodiment of the present invention is directed to a topical composition useful for the treatment of fungal, yeast and/or bacterial infections which includes a pharmaceutically effective amount of a carbohydrate, such as a sugar, to treat a patient in need of such treatment and a pharmaceutically acceptable carrier.

A further aspect of the present invention provides a method for the treatment of bacterial, yeast and/or fungal infections including administering a pharmaceutically effective amount of a composition having a carbohydrate, such as a sugar and a pharmaceutically acceptable carrier. Yet a further embodiment of the present invention provides a method for he treatment of bacterial, yeast and/or fungal infections including administering a pharmaceutically effective amount of a carbohydrate, such as a sugar, to the infection of a patient in need of such treatment. Still a further embodiment provides for a method for the treatment of pain associated with fungal, yeast and/or bacterial infections including administering a pharmaceutically effective amount of a carbohydrate such as a sugar to a patient in need of such treatment. Useful carbohydrates include simple and complex sugars. Useful sugars, include but are not limited to, sugars with three to nine carbons and may be either straight chain or cyclic, such as in the furanose or pyranose configurations. Useful sugars include but are not limited to sucrose, glyceraldehyde, dihydroxyacetone, erythrose, ribose, ribulose, xylulose, glucose, galactose, mannose, fructose, sedoheptulose, neuraminic acid also called sialic acid and isomers, enantiomers and combinations thereof.

### DETAILED DESCRIPTION

An aspect of the invention is directed to an antiyeast, antifungal or antibacterial composition that includes a pharmaceutically active agent or ingredient and a pharmaceutically acceptable carrier. A further embodiment of the present invention provides a method of treating yeast, bacterial or fungal infections of the skin, nails and mucous membranes by administering a composition having a pharmaceutically active agent and a pharmaceutically acceptable carrier. In multiple embodiments, there is provided a topical anti-yeast, anti-fungal and antibacterial composition including a carbohydrate and at least one additional pharmaceutically active agent. The pharmaceutically active agent may be a carbohydrate including simple and complex sugars. Useful sugars include ketones and aldehydes. Useful pharmaceutically active agents include sugars such as, dihydroxyacetone, glyceraldehydes and enantiomers and derivatives and combinations thereof.

Useful sugars include monosaccharides, disaccharides, trisaccharides, oligosaccharides, polysaccharides. Additionally, useful sugars and sugar derivatives include, but are not limited to, tagatose, sorbose, erythrose, erythrulose, sucralose sucrose, glyceraldehyde, dihydroxyacetone, erythrose, ribose, ribulose, xylulose, glucose, galactose, mannose, fructose, sedoheptulose, neuraminic acid also called sialic acid and isomers, enantiomers and combinations thereof.

In multiple embodiments, the pharmaceutically active agent is a hydroxyketone. Useful hydroxyketones include 2-hydroxy 1,4-naphthaquinone and dihydroxyacetone and derivatives and enantiomers thereof. In one embodiment, the hydroxyketone is dihydroxyacetone or an enantiomer or derivative thereof. Dihydroxyacetone (DHA) is a white, crystalline, hygroscopic powder having the chemical formula C₃H₆O₃.

In multiple embodiments, the pharmaceutical active agent is an aldehyde. Useful aldehydes include but are not limited to glyceraldehyde and derivatives and enantiomers thereof. Glyceraldehyde (GLA) has the chemical formula of C₃H₆O₃.

Useful derivatives include, but are not limited to, phosphorylated derivatives of ketones and aldehydes, such as dihydroxyacetone phosphate and 3-phosphoglyceraldehyde, 2-phosphoglyceraldehyde, 1:3 bisphosphoglycerate, 30-phosphoglycerate and enantiomers and combinations thereof.

Pharmaceutically active agents used in the various embodiments of the present invention are desirably present in a composition in a pharmaceutically effective amount for the treatment of pathological infections such as yeast, bacterial and fungal infections. Useful pharmaceutically effective amounts of the pharmaceutically active agent range from about 0.0001% to about 99% by weight, from about 0.005% to about 50% by weight, from about 0.005% to about 15% by weight and from about 0.005% to about 10% by weight. Particularly useful amounts of the pharmaceutically active agent range from about 0.005% to about 8% weight, from about 3% to about 8% weight. Useful amounts of glyceraldehyde or dihydroxyacetone include from about 0.005 to about 10% by weight, from about 2 to about 5% by weight, about 5% or about 2.5% by weight.

In several embodiments, there is provided a composition that includes at least one second skin agent and/or pharmaceutically active agent. Useful additional skin agents or pharmaceutically active agents include, but are not limited to analgesics, anesthetics, antibiotics, anti-psoriatic agents, biocides, botanicals, fungicides, insecticides, keratolytic agents, nail conditioners, nail growth agents, nutrients, sunscreens, vitamins and combinations thereof.

Useful second pharmaceutically active agents include but are not limited to miconazole, econazole, ketoconazole, itraconazole, fluconazole, bifoconazole, terconazole, butoconazole, tioconazole, oxiconazole, sulconazole, saperconazole, clotrimazole, butenafine, undecylenic acid, haloprogin, tolnaftate, nystatin, ciclopirox olamine, terbinafine, amorolfine, naftifine, elubiol, griseofulvin, corticosteroids, calcipotriene, anthraline, minoxidil, minoxidil sulfate, retinoids, cysteine, acetyl cysteine, methionine, glutathione, biotin, finasteride and ethocyn, tea tree oil, mupirocin, neomycin sulfate bacitracin, polymyxin B, 1-ofloxacin, chlortetracycline hydrochloride, oxytetracycline hydrochloride, tetrachcycline hydrochoride, clindamycin phsphate, gentamicin sulfate, benzatkonium chloride, benzethonium chloride, hexylresorcinol, methylbenzethonium chloride, phenol, quaternary ammonium compounds, triclocarbon, triclosan, vitamins, amino acids, betamethasone dipropionate, betamethasone valerate, clobetasol propionate, corticosteroids, hydrogen peroxide, sodium hyperchloridehydroxy acids, keto acids, urea, retinoids, glycols, glycol ethers, dimethyl sufoxide, caprolactam, benzocaine, pramoxine, lidocaine, dyclonine, bupivacaine, prilocaine, ropivacaine, and pharmaceutically acceptable salts thereof and combinations thereof.

Useful pharmaceutically active agents include, but are not limited to, botanical extracts of plants such as the silver birch (Betulla alba), or Eclipta alba which contains flavonoids known as wedelolactone, demethylwedelolactone, henna and combinations thereof. Such botanical sources are described in U.S. Pat. No. 5,559,146, which is incorporated herein by reference. One particular formulation is known as Mahakanni STLC, a liposome concentrate whose active ingredient(s) are believed to be derived from Eclipta alba.

A number of biocidal agents are useful with the present invention, including quarternary ammonium compounds, phenolic compounds and peroxygen compounds such as peroxy acids. Useful biocide agents include quaternary ammonium compounds, thymol and Triclosan® and combinations thereof. Preferably, Triclosan® is present at a concentration of about 0.9 to about 1.1% wt, or from about 1.0 wt-%. Triclosan® gives a broad spectrum of pathogenic coverage and has a long history of safe usage with a benign toxicological profile. Triclosan® is a chlorinated diphenyl ether. Hexetidine is also useful in the present invention.

Further useful pharmaceutically active agents include fungicidal agents such as morpholines, allylamines and triazoles. More particularly, useful fungicidal agents include amoroline, betadiene, bifonazole, butenafine, clotrimazole, econazole nitrate, isoconazole, ketoconazole, miconazole nitrate, naftifine hydrochloride, oxiconazole, sulconazole, sulfanazole, terbinafine, ticonazole, tolnaftate, undecenoates, ciclopirox, fluconazole and pharmaceutically acceptable salts thereof as well as combinations thereof. Another useful pharmaceutical agent is iodine. Useful imidazole and various imidazole derivatives include 4-(hydroxymethyl)imidazole (see U.S. Pat. No. 5,252,322 to Stoner et al.) and pyridine N-oxide and its derivatives (see U.S. Pat. Nos. 4,293,542 to Lang et al. and 4,228,151 to Lang et al.), which are all incorporated herein by reference.

Additional useful active pharmaceutical agents include topical analgesics and anesthetics, such as those described in U.S. Pat. No. 6,432,986, which is incorporated herein in its entirety. Examples of useful topical active pharmaceutical agents include, but are not limited to, doconosol, benzocaine, pramoxine, lidocaine, dyclonine, bupivacaine, prilocaine, ropivacaine and isomers and salts thereof and combinations thereof. Further examples of useful pharmaceutical active compounds/agents include but are not limited to retinoic acid, urea, ascorbic acid, propylene glycol, selenium sulfide, salicylic acid, pyrrolidone 5 carboxylic acid, hydrocortisone, betamethasone benzoate, desfluorotriamcinolone, triamcinolone acetonide, dexamethasone, dexamethasone acetate, flumethasone pivalate, flumethasone valerate, deprodone propionate, bufexemac, suprofen, tetracycline, oxytetracycline, chlorotetracycline, neomycin, erythromycin, bacitracin, streptomycin, chloromycetin, griseofulvin, mycostatin, miconazole, miconazole nitrate, metholtrexate, chlorhexidine, domiphen bromide, benzalkonium chloride, cetyl pyridinium chloride, dequalinium chloride, cetyl trimethyl ammonium bromide, benzethonium chloride, methylbenzethonium chloride, 3,4,4′-trichlorocarbanilide, 3,4,5-tribromosalicylanilide, dichlorophene, tetrachlorophene, hexachlorophene, 2,4,4′-trichloro-2′-hydroxydiphenylether, zinc pyrithione, iodine and the iodophores derived from non-ionic surface active agents and from polyvinylpyrrolidone and isomers and enantiomers thereof and combinations thereof.

Further embodiments provide for compositions including a penetration enhancer and applications of such compositions. Nail plates have a high sulphur content in the form of disulfide bonds. U.S. Pat. No.5,696,164, incorporated herein in its entirety, discloses the use of thio-containing amino acids and its derivatives (i.e., sulfhydryl-containing amino acids), such as cysteine and N-acetyl cysteine, and urea to increase drug permeability in a nail plate, by breaking disulfide bonds in nail keratin to increase drug penetration into and through the nail. Useful nail-penetration agents, include but are not limited to, glycols, glycol ethers, dimethyl sulfoxide, caprolactam, and other hydrophilic compounds to facilitate the penetration of allylamine fungicides into the nail, as disclosed in European Patent Application EP 503988 A1 (1992), incorporated herein in its entirety. A penetration enhancer may include any agent that facilitates the composition to travel into the nailbed via capillary action. Useful penetration enhancers include viscosity altering agents to lower the viscosity composition.

Still further embodiments provide for a composition including a keratinization agent to facilitate the nail to reattach to the nailbed and a method of keratinizing the infected nailbed to facilitate the reattachment of the nail to the nail bed by applying a composition with a keratinization agent. Suitable examples of keratolytic agents, e.g. an active agent having desquamating, exfoliant, or scrubbing properties, or an active agent which can soften the horny layer of the skin; include but are not limited to alpha (α) and/or beta (β) hydroxy acids; benzoyl peroxide; keto acids, such as pyruvic acid, 2-oxopropanoic acid, 2-oxobutanoic acid, and 2-oxopentanoic acid; oxa acids, as disclosed in U.S. Pat. Nos. 5,847,003 and 5,834,513, the disclosures of which are incorporated herein by reference; urea; retinoids, or any combinations thereof. These agents can be formulated, for example, in amounts of from about 0. 0001% to 20% by weight relative to the total weight of the composition. More specifically, examples of hydroxy acids include, but are not limited to, α-hydroxy acids or β-hydroxy acids, either linear, branched, cyclic, saturated or unsaturated. The hydrogen atoms in the carbon-based backbone of these materials can be substituted with halogens, halogen-containing alkyl, acyl, acyloxy, alkoxycarbonyl, or alkoxy radicals having from 2 to 18 carbon atoms. Suitable hydroxy acids include, for example, glycolic acid, lactic acid, malic acid, tartaric acid, citric acid, 2-hydroxyalkanoic acid, mandelic acid, salicylic acid and alkyl derivatives thereof, including 5-n-octanoylsalicylic acid, 5-n-dodecanoylsalicylic acid, 5-n-decanoylsalicylic acid, 5-n-octylsalicylic acid, 5-n-heptyloxysalicylic acid, 4-n-heptyloxysalicylic acid and 2-hydroxy-3-methylbenzoic acid or alkoxy derivatives thereof, such as 2- hydroxy-3-methyoxybenzoic acid. Exemplary salicylates include methyl salicylate, acetyl salicylate, acetaminophen and combinations thereof. Exemplary retinoids include, without limitation, retinoic acid (e.g., all-trans or 13-cis) and derivatives thereof, retinol (Vitamin A) and esters thereof, such as retinol palmitate, retinol acetate and retinol propionate, and salts thereof. Preferred exfoliation promoters are 3,6,9-trioxaundecanedioic acid, glycolic acid, lactic acid, or any mixtures thereof. Other acids, such as oxa acid (e.g., U.S. Pat. No. 6,069,169) and an oxa diacid (e.g., U.S. Pat. No. 5,932,229) can be included in the compositions of this invention.

Peroxy compounds may also be included to condition the skin to improve the efficacy of the compositions. Useful peroxy compounds include hydrogen peroxide, sodium hyperchloride and the like.

Examples of antipsoriatic drugs useful in the present invention include but are not limited to corticosteroids (e.g., betamethasone dipropionate, betamethasone valerate, clobetasol propionate, diflorasone diacetate, halobetasol propionate, amcinonide, desoximetasone, fluocinonide, fluocinolone acetonide, halcinonide, triamcinolone acetate, hydrocortisone, hydrocortisone verlerate, hydrocortisone butyrate, aclometasone dipropionte, flurandrenolide, mometasone furoate, methylprednisolone acetate), calcipotriene and anthraline. Specific antipsoriatic drugs include betamethasone dipropionate, betamethasone valerate, and clobetasol propionate.

Useful nail growth promoters include but are not limited to minoxidil, minoxidil sulfate, retinoids, cysteine and acetyl cysteine, methionine, glutathione, biotin, finasteride and ethocyn, as well as pharmaceutically acceptable salts of these compounds. The preferred growth promoter are minoxidil, minoxidil sulfate, retinoids, cysteine and acetyl cysteine. The particularly preferred nail growth promoters are 2% minoxidil, 2% minoxidil sulfate, and 0.1% retinol.

Useful nutrients may also be utilized and include but are not limited to vitamins, amino acids, and their derivatives. Examples of such nutrients include but are not limited to vitamin B complex: thiamine, nicotinic acid, biotin, pantothenic acid, choline riboflavin, vitamin B 6, vitamin B 12, pyridoxine, inositol, carnitine; ascorbic acid, ascorbyl palmitate, vitamin A, vitamin K, vitamin E, vitamin D, cysteine and N-acetyl cysteine, herbal extracts, and their derivatives.

Nail conditioners may also be utilized and include but are not limited to mineral-containing compounds, flavonoids and retinoids. These nail conditioners improve general nail conditions, such as strengthening the nails to prevent nail chipping and cracking, and to beautify the nails. Examples of such agents include but are not limited to calcium pantothenate, calcium carbonate, and calcium gluconate. Examples of retinoids include but not limited to retinol (Vitamin A alcohol), retinal (Vitamin A aldehyde), retinyl acetate, etinyl palmitate, retinoic cid, 9-cis-retinoic acid and 13-cis-retinoic acid. When retinoids are the active agents, the concentration of retinoids is from about 0.01% to about 0.5%, preferably, from about 0.05 to about 0.1%. Examples of flavonoids include but not limited to naringenin, quercetin, catechins (e.g., epigallocatechin gallate), theaflavins, robustaflavone, hinokiflavone, amentoflavone, agathisflavone, volkensiflavone, morelloflavone, rhusflavanone, and succedangeaflavanone.

In one embodiment, the present invention provides an acidified composition including at least one active agent, at least one acidifier, at least one volatile solvent, and at least one active agent. The term “acidifier” refers to substances which are liquids having an apparent pH of ≦1, or solids having a pKa≦5 as disclosed in U.S. Pat. No. 6,231,875, which is incorporated herein in its entirety. Apparent pH is the pH reading measured by a glass pH electrode. Useful acidifiers include 37% HCl, 10% HCl, sulfuric acid, o-phosphoric acid, nitric acid, acetic acid, L (+)-lactic acid, salicylic acid, and glycolic acid. Particularly preferred acidifiers include 37% HCl and 10% HCl. If the total weight of the acidified composition is 100 parts, the acidifier should be about 0.05 to about 50% w/w (w/w=weight/weight), or from about 0.1% to about 10%, or from about 0.5% to about 5%.

Another embodiment of the present invention provides for a buffered composition. Useful buffers include but are not limited to phosphate, borate and acetate buffers. Useful buffered compositions may have a pH in the amount range from about 5.0 to about 6.0.

Multiple embodiments of the present invention may be applied to diseased skin, nails or mucous membranes around the human or an animal body. Various embodiments of the present invention are able to treat the diseased skin, nail and mucous membranes in a matter of several days. In fact, improvement of bacterial and fungal infections has been noticed in as little time as after about one week of applications. Although the color of the nail due to the infection may not disappear immediately upon treatment, the new nail and skin growth exhibit a healthy, clear and thin nail appearance. The pain associated with the bacterial or fungal infection has been observed to be reduced, minimized, or disappear upon or shortly thereafter treatment.

Treatment with the pharmaceutically active agent includes a dosing regimen of one, two or three applications per day to the infected area for a period of one day to three consecutive weeks. However, shorter and longer treatment or more frequent applications per day may be appropriate according to the severity of the infection. Another embodiment of the present invention provides for maintenance program for periodic applications such as one application every one to two weeks after the initial treatment. Another embodiment of the present invention provides for a method of preventing nail, skin and mucous membrane infections such as fungal, bacterial and yeast infections. The application should be directly applied on the infected skin area, on the infected nail area or on the nail bed underneath the nail proper. After the initial treatment of seven days, it has been observed that no additional treatments may be necessary. Because toe nails grow very slowly, it typically takes 6 months to a year for the nail to completely regain a healthy, clear, thin appearance; although clear evidence of continued improvement can be seen prior to that time.

The topical treatment of the compositions of the present invention may be employed in combination with systemic oral or topical treatment. For example, an antifungal drug, such as, itraconazole, terbinafine, griseofulvin, miconazole, fluconazole, isomers and derivatives thereof or other antifungal drugs, can be given orally over a period of time. This time period may be concurrent during the entire topical treatment regimen, or concurrently during a portion of the topical treatment regimen, or before or after the topical treatment.

The invention further includes a method of treating healthy human nails or skin by topically applying compositions of the present invention. The compositions of the present invention may be used prophylactically to prevent infection. Typically the prophylactic treatment regimen for fungal and/or bacterial infections of the nail and skin using the composition can vary from once or twice per day or week to once or twice per month, with the interval between treatments shorter for the skin and longer for the nail.

Multiple embodiments of the present invention are effective in killing, substantially inhibiting the growth and preventing or reducing the occurrence of pathological infections such as fungi, yeast and bacteria, including but not limited to dermatophytes or yeast, epidermophyton, microsporum, trichophyton rubrum, trichophyton mentagrophytes , candida albicans, andropophiles, zoophiles, geophiles and combinations thereof. The present invention is also useful in treating nail and skin diseases such as onychomycosis, psoriatic nails, psoriasis of the skin, versicolor, ringworm, plantar tinea pedis, Jock itch, and athlete's foot.

Various embodiments of the invention include compositions in formulations such as lotions, topical creams, mousses, ointments, solutions, liquids, sprayable liquids, emulsions, lacquers, gels, stand-alone single layer films, multiple layer films, bioadhesive films, patches, bandages with an ointment or gel attached thereto, nail polish and combinations thereof. One embodiment provides a nail polish with the compositions of the present invention. Another embodiment of the present invention provides for a standalone film including a water soluble polymer, such as pullulan and the like, with a pharmaceutically active agent for application directly to the infected area. In this embodiment, the film is placed on the infected area and disintegrate by the moisture provided by the body part of the infected area or by an external source of moisture.

Another embodiment of the present invention provides for a kit or product including a composition of the present invention in a package. Suitable packages include hard tubes, squeezable flexible tubes, non-aerosolized sprays, aerosolized sprays, containers with integrated pumps, dispensers with an integrated brush or foam attached thereto. Useful brushes include brushes with hard bristles to provide an exfoliation effect on the skin or soft brushes. Tubes may be flexible squeezable tubes or hard tubes with an appropriate applicator. Appropriate applicators include a spray, brush, felt pad, foam pad, dauber applicator, rubber pad, sponge pad, roll on applicator, stick applicator and pen applicator and combinations thereof.

The term “pharmaceutically or cosmeceutically acceptable” as used herein refers to materials that are not known to be harmful to humans. These materials can be found for example in the CTFA International Cosmetic Ingredient Dictionary 4th Edition, The Cosmetic, Toiletry, and Fragrance Association, Inc., Washington, D.C., 1991, FDA's Inactive Ingredient Guide, as well as in Remington's Pharmaceutical Sciences, 18th Edition, A. R. Greenaro Ed., Mack Publishing Co., Easton, Pa., 1990, all of which are incorporated herein by reference. Suitable cosmeceutically or pharmaceutically acceptable carriers include, but are not limited to solutions, especially hydroalcoholic solutions; suspensions; emulsions, especially oil-in-water emulsions, most especially nonionic oil-in-water emulsions; gels, mousses, patches, aerosols and the like. The specific type of carrier used will vary with the desired physical, aesthetic and pharmacological properties of the final composition.

Various other optional ingredients may be included in the compositions of the present invention, including but not limited to emulsifiers, stabilizers, preservatives, emollients, antiseptics, pigments, dyes, humectants, moisturizers, propellants, and sunscreens, as well as other classes of materials whose presence may be cosmetically, or pharmaceutically desirable. Common examples of such ingredients are provided below by way of example and not limitation. Optional ingredients include polyethylene glycols such as Peg-100 stearate, glyceryl monostearate, DEA cetyl phosphate, dimethicone copolyol, TEA stearate and the like; polyols such as glycerine and propylene glycol, hydrocarbons such as mineral oil and petrolatum, fatty acid esters such as myristyl lactate and caprylic and capric triglycerides, silicones, and natural whole oils or components thereof, wheat lipid extracts or ceramides, methyl paraben, ethyl paraban, methyl dibromoglutonylnitrile, butyl paraben, propyl paraben and phenoxyethanol, and derivatives and combinations thereof.

### EXAMPLE 1

Solutions of 5% and 10% dihydroxyacetone (DHA) were prepared in a 10% by weight glycerin in water emulsion. The solutions were tested against fungi and bacterial growth as shown in Table 1.

TABLE 1
Minimum Inhibitory Concentrations (MIC's) for 5% and 10% solutions of
DHA in 10% Glycerin vs. Fungi and Bacterial Organisms

| Composition | C. albicans (fungi) | S. aureus (bacteria) |
|---|---|---|
| 5% DHA in 10% Glycerin, | 50% | 12.5% |
| 85% Deionized water | | |
| 10% DHA in 10% Glycerin, | 25.0% | 6.25% |
| 80% Deionized water | | |

As shown in Table 1, the DHA solutions were effective in reducing the fungal and bacterial organism growth.

### EXAMPLE 2

Patient A is a male that had a fungal infection located in the toe nail area for over 12 years. The fungal infection had caused the nail of the patient to become thick, disfigured and yellowish in color and caused the patient a great deal of pain in the foot area especially when the patient attempted physical activity such as walking or running. Patient A had the clinically diagnosed fungal infection for several years and unsuccessfully used the currently available treatments including oral Lamisil®, iodine and several other therapies.

Composition 1, 5% DHA as shown in Table 2, was administered to the patient by topically applying it to the toe nails of patient A with the fungal infection one time a day for three consecutive weeks. Pain associated with the infection completely disappeared within seven days of treatment. Patient A was able to perform physical activity such as running without experiencing any pain in the toe nail area. The thickened disfigured toe nail by the fungal infection did not disappear, however, no discoloration or thickening was observed in the new nail growth after treatment with DHA. The nail specimen was dermatophyte free upon culture. The patient continues a maintenance treatment schedule of one application every two weeks until the entire nail grows out completely.

TABLE 2

| | % Weight | | | |
|---|---|---|---|---|
| Ingredient | Composition 1 | Composition 2 | Composition 3 | Composition 4 |
| Dihydroxyacetone DHA) | 5 | | 5 | |
| Glyceraldehyde | | 2.5 | | 2.5 |
| Glycerol | 10 | 10 | 10 | 10 |
| Deionized Water | 85 | 87.5 | 85 | 87.5 |
| 0.05 M Acetate Buffer | | | Adjusted to | Adjusted to |
| adjusted to pH 6.0 | | | pH 6.0 | pH 6.0 |

### EXAMPLE 3

Patient B is a male that had lost the toe nail due to an accident and the new nail growth developed thickened and disfigured nail. The nail separated from the nail bed. The patient was treated once daily with composition 4 for one month. Composition 4 is a glyceraldehyde (GLA) 2.5% composition that is adjusted to a pH of 6.0 with 0.05 Molar (M) sodium acetate buffer and titrated with HCl and/or NaOH, as appropriate. Following the treatment, the associated pain with the condition disappeared and the nail specimen was negative for dermatophytes upon culture.

### EXAMPLE 4

The minimum inhibitory concentration (MIC) of compositions 1 and 2 detailed in Table 2, against thirty dermatophyte isolates was determined according to the modification of NCCLS method for susceptibility testing of dermatophytes developed at the Center for Medical Mycology. MIC testing was performed in RPMI 1640 as a medium, 35° C. and 4 days as incubation temperature and time, and 1-3×10³ conidia/ml as inoculum size. The MIC endpoint was defined as the lowest concentration that inhibited 80% of fungal growth as compared to the growth control.

Ten isolates of the following microorganisms were tested: Trichophyton rubrum, Trichophyton mentagrophtyes and Epidermophyton floccosum

### RESULTS

The MIC₅₀ (defined as the minimum concentration to inhibit 50% isolates tested) of compound 18638-8 against all dermatophytes was 1.56 mg/ml. The MIC₉₀ (defined as the minimum concentration to inhibit 90% of the isolates tested) of compound 18638-8 against all dermatophytes was 3.125 mg/ml.

The MIC₅₀ (defined as the minimum concentration to inhibit 50% isolates tested) of composition 1 against all dermatophytes was 0.906 mg/ml. The MIC₉₀. (defined as the minimum concentration to inhibit 90% of the isolates tested) of composition 2 against all dermatophytes was 1.813 mg/ml. Table 3 summarizes the data.

TABLE 3
Composition 1 (in mg/ml)

| Organism | MIC₅₀ | MIC₉₀ |
|---|---|---|
| T. rubrum | 1.560 | 0.780 |
| E. floccosum | 0.780 | 1.560 |
| T. mentagrophytes | 1.560 | 3.125 |
| All dermatophytes | 1.560 | 3.125 |

Composition 2 (in mg/ml)

| Organism | MIC₅₀ | MIC₉₀ |
|---|---|---|
| T. rubrum | 0.453 | 0.453 |
| E. floccosum | 0.906 | 0.906 |
| T. mentagrophytes | 1.813 | 1.813 |
| All dermatophytes | 0.906 | 1.813 |

While the invention has been described in detail and with reference to specific examples thereof, it will be apparent to one skilled in the art that various changes and modifications can be made therein without departing from the spirit and scope thereof.

What is claimed is:

1. A topical composition useful for the treatment of yeast, fungal or bacterial infections of a patient in need of such treatment comprising a pharmaceutically effective amount of a anti-yeast, anti-fungal or antibacterial pharmaceutically active agent selected from the group consisting of ketones, aldehydes and enantiomers, derivatives and combinations thereof and a pharmaceutically acceptable carrier.

2. The composition of claim 1, wherein said pharmaceutically active agent is selected from the group consisting of dihydroxyacetone, dihydroxyacetone phosphate, glyceraldehyde, 3-phosphoglyceraldehyde and enantiomers, derivatives and combinations thereof.

3. The composition of claim 2, wherein said pharmaceutically active agent is selected from the group consisting of dihydroxyacetone, dihydroxyacetone phosphate and enantiomers, derivatives and combinations thereof.

4. The composition of claim 2, wherein said pharmaceutically active agent is selected from the group consisting of glyceraldehyde, 3-phosphoglyceraldehyde and enantiomers, derivatives and combinations thereof.

5. The composition of claim 2, wherein said pharmaceutically active agent is present in an amount from about 0.005% to about 50% by weight.

6. The composition of claim 2, wherein said pharmaceutically active agent is present from about 0.005 to about 10% by weight.

7. The composition of claim 2, wherein said pharmaceutically active agent is present from about 0.005 to about 5% by weight.

8. The composition of claim 2, further comprising at least one additional pharmaceutically active agents selected from the group consisting of analgesics, anesthetics, antibiotics, anti-psoriatic agents, biocides, botanicals, fungicides, insecticides, keratolytic agents, nail conditioners, nail growth agents, nutrients, sunscreens, vitamins and combinations thereof.

9. The composition of claim 2, further comprising at least one additional pharmaceutical active agent selected from the group consisting of wherein said at least one active agent is selected from the group consisting of miconazole, econazole, ketoconazole, itraconazole, fluconazole, bifoconazole, terconazole, butoconazole, tioconazole, oxiconazole, sulconazole, saperconazole, clotrimazole, butenafine, undecylenic acid, haloprogin, tolnaftate, nystatin, ciclopirox olamine, terbinafine, amorolfine, naftifine, elubiol, griseofulvin, corticosteroids, calcipotriene, anthraline, minoxidil, minoxidil sulfate, retinoids, cysteine, acetyl cysteine, methionine, glutathione, biotin, finasteride and ethocyn, tea tree oil, mupirocin, neomycin sulfate bacitracin, polymyxin B, 1-ofloxacin, chlortetracycline hydrochloride, oxytetracycline hydrochloride, tetrachcycline hydrochoride, clindamycin phsphate, gentamicin sulfate, benzalkonium chloride, benzethonium chloride, hexylresorcinol, methylbenzethonium chloride, phenol, quaternary ammonium compounds, triclocarbon, triclosan, vitamins, amino acids, betamethasone dipropionate, betamethasone valerate, clobetasol propionate, corticosteroids, hydrogen peroxide, sodium hyperchloridehydroxy acids, keto acids, urea, retinoids, glycols, glycol ethers, dimethyl sufoxide, caprolactam, benzocaine, pramoxine, lidocaine, dyclonine, bupivacaine, prilocaine, ropivacaine, and pharmaceutically acceptable salts thereof and combinations thereof.

10. The composition of claim 1, further comprising at least one second pharmaceutical active agent selected from the group consisting of terbinafine, fluconazole, micanozole, doconosol and derivatives, enantiomers, pharmaceutically acceptable salts and combinations thereof.

11. The composition of claim 1, further comprising fluconazole or pharmaceutically acceptable salt thereof.

12. The composition of claim 1, wherein said composition is a formulation selected from the group consisting of lotions, topical creams, mousses, ointments, solutions, liquids, sprayable liquids, emulsions, lacquers, gels, stand-alone single layer films, multiple layer films, bioadhesive films, patches, bandages with an ointment or gel attached thereto, nail polish and combinations thereof.

13. A product comprising the composition of claim 1, wherein said product is in a package selected from the group consisting of a hard tube, squeezable flexible tube, non-aerosolized spray, aerosolized spray, containers with integrated pump, dispensers with an integrated brush or foam attached thereto.

14. A method for the treatment of yeast, bacterial or fungal infections comprising administering a pharmaceutically effective amount of a pharmaceutically active agent selected from the group consisting of a ketones, aldehydes and derivatives, enantiomers and combinations thereof to a patient in need of such treatment.

15. The method of claim 14, wherein said pharmaceutically active agent is selected from the group consisting of dihydroxyacetone, dihydroxyacetone phosphate, glyceraldehyde, 3-phosphoglyceraldehyde and enantiomers, derivatives and combinations thereof.

16. The method of claim 15, wherein said pharmaceutically active agent is administered topically to infected skin, nail or mucous membranes.

17. The method of claim 15, wherein said pharmaceutically active agent is present in an amount from about 0.005% to about 15%.

18. The method of claim 14, further comprising concurrently administering at least one pharmaceutically active agent selected from the group consisting of terbinafine, fluconazole, micanozole, doconosol and a derivatives, enantiomers, pharmaceutically acceptable salts and combinations thereof.

19. The method of claim 14, wherein said composition is administered via a brush, spray, felt pad, foam pad, dauber applicator, rubber pad, sponge pad, roll on applicator, stick applicator, pen applicator and combinations thereof.

20. A composition comprising at least one first pharmaceutically effective amount of a pharmaceutically active agent selected from the group consisting of dihydroxyacetone, dihydroxyacetone phosphate, glyceraldehyde, 3-phosphoglyceraldehyde and enantiomers, derivatives and combinations thereof and at least one second pharmaceutically active agent in a pharmaceutically effective amount selected from the group consisting of terbinafine, fluconazole, micanozole, doconosol and a derivative, enantiomer, pharmaceutically acceptable salt and combination thereof; and a pharmaceutically acceptable carrier.
